(19) United States
(12) Patent Application Publication
(10) Pub. No.: US 2021/0363541 A1
(43) Pub. Date: Nov. 25, 2021

(54) PREVENTION OR TREATMENT OF CARDIAC ARRHYTMIA AND SUDDEN CARDIAC DEATH

(71) Applicant: GEORG-AUGUST-UNIVERSITÄT GÖTTINGEN STIFTUNG ÖFFENTLICHEN RECHTS, UNIVERSITÄETSMEDIZIN, Goettingen (DE)

(72) Inventors: Maria Patapia ZAFEIRIOU, Goettingen (DE); Laura ZELARAYAN, Goettingen (DE); Wolfram Hubertus ZIMMERMANN, Goettingen (DE)

(73) Assignee: GEORG-AUGUST-UNIVERSITÄT GÖTTINGEN STIFTUNG ÖFFENTLICHEN RECHTS, UNIVERSITÄTSMEDIZIN, Goettingen (DE)

(21) Appl. No.: 16/975,298

(22) PCT Filed: Feb. 21, 2019
(86) PCT No.: PCT/EP2019/054270

§ 371 Date: Aug. 24, 2020

(30) Foreign Application Priority Data
Feb. 22, 2018 (GB) 1802864.7

(51) Int. Cl. C12N 15/86; A61K 38/17; A61K 41/00; A61P 9/06

(52) U.S. Cl. CPC C12N 15/86; A61K 38/17; C12N 2750/14171; A61P 9/06; C12N 2750/14143; A61K 41/0028

(57) ABSTRACT

The present disclosure concerns agents for use in a new therapeutic application for the prevention or treatment of cardiac arrhythmia and sudden cardiac death. More specifically, the present disclosure concerns an expression construct capable of enhancing expression of TBX5 in a subject to be treated, for use in the prevention or monotherapeutic treatment of a ventricular heart disease and associated complications selected from cardiac arrhythmia and sudden cardiac death.

The present disclosure concerns agents for use in a new therapeutic application for the prevention or treatment of cardiac arrhythmia and sudden cardiac death. More specifically, the present disclosure concerns an expression construct capable of enhancing expression of TBX5 in a subject to be treated, for use in the prevention or monotherapeutic treatment of a ventricular heart disease and associated complications selected from cardiac arrhythmia and sudden cardiac death.

### BACKGROUND OF THE INVENTION

Cardiovascular disease (CVD) is the number one cause of death worldwide (World Health Organization factsheets 09/2016). More than 60% of all deaths due to CVDs are represented by out-of-hospital Sudden Cardiac Death (SCD)(1). Pharmacological heart failure treatments aim at protecting the heart from neurohumoral overstimulation, which may trigger fatal arrhythmias.

T-box 5 (TBX5) is an essential transcription factor for cardiac development (2). In humans, TBX5 mutations cause Holt-Oram Syndrome (HOS), a rare autosomal congenital disease linked to abnormal cardiac electrophysiology and arrhythmias (3). A genome wide association study (GWAS) revealed an association between alterations in the TBX5 locus and atrial fibrillation, atrioventricular block and QRS-prolongation (4).

Zhou et al. International Journal Of Molecular Medicin, 36: 282-288 (2015) (32) disclose a TBX5 loss-of-function mutation associated with sporadic dilated cardiomyopathy.

In line with the human data, heterozygous TBX5 knockout (KO) mice exhibit a similar phenotype as observed in patients with HOS, including conduction defects, heart and limb malformations (5). Specific deletion of TBX5 in the adult ventricular conduction system (VCS) resulted in reduced Nav1.5 and CX40 expression, loss of fast conduction, arrhythmias and SCD (6). In the adult atria, inducible TBX5 loss of function caused primary spontaneous and sustained atrial fibrillation (7). While the role of TBX5 for normal electrophysiological function in atrial tissue and the VCS is well established, it remains unclear whether TBX5 in adult ventricular cardiomyocytes (CMs) plays a critical role for cardiac homeostasis.

WO 2013/063305 A2 discloses Tbx5 in the context of a method for directed cardiomyocyte differentiation of stem cells.

WO 2013/173714 A2 discloses a method of making a fast conducting cardiomyocyte comprising (a) obtaining a cardiomyocyte; and (b) increasing TBX5 in the cardiomyocyte thereby converting the cardiomyocyte into a fast conducting cardiomyocyte.

WO 2014/071199 A1 discloses a method of angiogenic conditioning to enhance cardiac cellular reprogramming of fibroblasts of infarcted myocardium, which uses a combination of angiogenic proteins and cardio-differentiating transcription factors.

There is still a need in the art for new or alternative therapeutic applications for the prevention or treatment of cardiac arrhythmia and sudden cardiac death.

### SUMMARY OF THE INVENTION

The inventors found that TBX5 protein abundance is significantly lower in left ventricular biopsies of patients with human ischemic heart disease and dilated cardiomyopathies when compared to non-failing hearts. Therefore, the inventors hypothesized that in spite of its relatively lower expression in ventricular CMs, TBX5 may play an important role in the adult working myocardium. To investigate the impact of TBX5 loss in cardiac function and electrical signal propagation in the ventricles, the inventors generated an inducible ventricular CM-specific Tbx5 knock out model (vTbx5KO). Finally, the inventors tested the therapeutic potential of TBX5 level normalization in vTbx5KO mice using an adeno-associated virus (AAV) vector.

In light of the experimental evidence presented in the examples, the present invention is directed to an expression construct capable of enhancing expression of TBX5 in a subject to be treated, for use in the prevention or monotherapeutic treatment of a ventricular heart disease and associated complications selected from cardiac arrhythmia and sudden cardiac death, as further defined in the claims.

### DETAILED DESCRIPTION OF THE PREFERRED EMBODIMENTS

The present disclosure is directed to an expression construct capable of enhancing expression of TBX5 in a subject to be treated, for use in the prevention or monotherapeutic treatment of a ventricular heart disease and associated complications selected from cardiac arrhythmia and sudden cardiac death.

Likewise disclosed is the use of an expression construct capable of enhancing expression of TBX5 in a subject to be treated in the preparation of a medicament for the prevention or monotherapeutic treatment of a ventricular heart disease and associated complications selected from cardiac arrhythmia and sudden cardiac death.

Further disclosed is a method for the prevention or monotherapeutic treatment of a ventricular heart disease and associated complications selected from cardiac arrhythmia and sudden cardiac death, wherein the method comprises the step of administering a subject to be treated an expression construct capable of enhancing expression of TBX5 in said subject.

As demonstrated in the examples below, the present inventors found that TBX5 protein abundance is significantly lower in left ventricular biopsies of patients with human ischemic heart disease and dilated cardiomyopathies when compared to non-failing hearts. Accordingly, the expression contruct of the present invention is intended to normalize the expression of TBX5 in the left ventricular heart. Thus, in a preferred embodiment, the expression construct is for normalizing the expression of TBX5 in ventricular cardiomyocytes.

In this context, the term “capable of enhancing expression of TBX5” as used herein is intended to mean that the expression construct, once it is delivered to the left ventricular heart, is capable of enhancing/increasing the TBX5 expression as compared to the TBX5 expression prior to administration of the expression construct of the present disclosure, and/or the average TBX5 expression of a group of corresponding subjects suffering from a ventricular heart disease and associated complications selected from cardiac arrhythmia and sudden cardiac death. The expression level and its increase/enhancement is preferably determined using quantitative RT-PCR, wherein the expression level of TBX5 is normalied to a house-keeping gene such as Gapdh. For example, TBX5 expression in humans with dilated cardiomyopathy and ischemic cardiomyopathy is about 0.1-0.4 (i.e. 10-40%) of the GAPDH expression in biopsy samples, wherein non-failing hearts show an expression of 0.8 (80%) of the GAPDH expression. As shown in FIG. 8A, the expression of TBX5 can be enhanced and increased by using an expression construct capable of enhancing expression of TBX5 to a value of 0.8-1.6 (80-160%). Thus, an expression level would be considered as being is normalized, if the expression of TBX5 was enhanced to be in the range of 0.5 to 1.6 (50-160%) as normalized to GAPDH, preferably in the range of 0.7 to 1.2 (70-120%), such as 0.8 to 1.0 (80-100%) as normalied to GAPDH. Further guidance how to carry out RT-PCR and suitable primer sequences are disclosed in the examples section below. Briefly, RNA is isolated using the NucleoSpin® RNA kit (Macherey-Nagel, Dueren/Germany) according to the manufacturer's instructions. Reverse transcription and quantitative PCR (qRT-PCR) are performed as described previously (19). Suitable primer sequences for murine TBX5 and murine GAPDH are listed in the Table 1 below. Suitable primers for human TBX5 can be designed on the basis of the sequence for human TBX5 (UNIPROT U89353.1) and suitable primer for GAPDH can be designed on the basis of the sequence of human GAPDH (UNIPROT P04406) using a primer designing software such as Primer3 (http://bioinfo.ut.ee/primer3/). Preferably the sequence framed by the primers contains 100-200 nucleotides and is in the 3′-end of the indicated sequences such that only fully transcribed RNAs are detected. Alternatively, commercial primer sets may be used, for example, the primer set for human GAPDH from Biomol (cat. No. VHPS-3541) and the primer set for human TBX5 from Sino Biological (cat. No. HP102800).

As noted above, the therapeutic application of the present disclosure is particularly suitable for a subject to be treated which subject shows a reduced expression of TBX5 in ventricular cardiomyocytes. In certain embodiments, the subject to be treated suffers from heart insufficiency, for example from dilated cardiomyopathy and/or ischemic cardiomyopathy.

The subject to be treated may be any kind of mammal such as a horse, cow, pig, mouse, rat, guinea pig, cat, dog, goat, sheep, non-human primate, or a human. However, in a particularly preferred embodiment, the subject to be treated is a human.

As will be described in more detail below, expression of TBX5 can principally be enhanced by at least three different ways: (1) by direct expression of TBX5 in ventricular cardiomyocytes (e.g. by using TBX5 expression constructs), (2) by enhancing endogenous TBX5 expression (e.g. by using the CRISPR-dCas9 activation system), or (3) by inactivating endogenous inhibitors of TBX5 expression (e.g. by inhibiting/inactivating microRNA-10a).

In one preferred embodiment, the expression construct encodes and/or is capable of expressing TBX5. The amino acid sequence of TBX5 and its coding nucleotide sequence are known and publicly derivable/available from gene bank data bases (651 entries in the NCBI database for mammal Tbx5). The coding sequence is preferably functionally linked with regulatory transcriptional and/or translational elements which are functional in the cardiomyocytes of the subject to be treated. In one embodiment, the expression contruct comprises Tbx5 in an expression cassette, as generally known in the art. Alternatively, the expression contruct may be in a form, e.g. in a form of a mRNA, which is not transcribed but translated into TBX5. Such an expression contruct may only require translational regulatory elements.

Thus, the expression contruct may be in the form of a recombined DNA, cDNA, RNA or modified RNA. Likewise, the expression contruct may be in the form of an expression vector. In a preferred embodiment, the expression construct is a viral expression vector. For example, one can use a lentiviral expression vector. However, since lentiviral vectors are integrating vectors, their use harbours a higher risk for carcinogenesis. Therefore, in a more preferred embodiment, the viral vector is an Adeno-associated viral (AAV) expression vector, which commonly provide for episomal expression. Of the 13 known serotypes, MV 1, 6, 8 and 9 have demonstrated cardiac tropism. The greatest cardiac tropism in most large animal models was demonstrated with AAV6 and AAV9. In humans AAV6 is the most efficient MV expression vector. See also Katz et al. (23), which is incorporated herein in its entirety by reference. Hence, in a preferred embodiment, the viral vector is an AAV serotype 6 vector, MV serotype 9 vector, MV serotype 1 vector, or MV serotype 8 vector, preferably an MV serotype 6 vector or MV serotype 9 vector, more preferably an AAV serotype 6 vector. In still another alternative, one may also use an adenovirus as the expression construct. Adenoviruses have a higher packaging capacity, and thus represent an advantage over the dCAS9 enhancer system described below which is relatively big. Further details with regard to recombinant adenovirus can be found in Lai et al. (33), which is incorporated herein in tis entirety by reference.

While in principle the expression of TBX5 may be controlled by using a constitutively expressing promoter, it is more preferred that the TBX5 expression is under the control of an inducible promoter, or a tissue-specific promoter. Of particular interest for use in the present invention are cardiac-specific promoter, in order to avoid bystander or side-effects in other organs. Suitable promoters may be selected, for example, from cardiac troponin T promoter (cTnT), α-myosin heavy chain (α-MHC), and myosin light chain (MLC2v). See also Lee et al. (24), which is incorporated herein in its entirety by reference. Particularly preferred is the embodiment, wherein the expression construct comprises human cardiac troponin-T promoter. Suitability of this promoter has been demonstrated in the examples below.

However, as noted above, expressing TBX5 is only one possible way to enhance expression of TBX5. In another embodiment, the expression construct encodes and/or is capable of expressing a CRISPR-dCas9-activator system specific for Tbx5. This system fuses a deactivated Caspase 9 (dCas9) with a transcriptional activator complex (VPR or SAM). Guided by gene-specific designed guide RNAs, here Tbx5 specific guide RNAs, the activator complex binds on promotor regions of the target gene, here Tbx5, and enhances endogenous transcription. The technique and its advantages are further described in the review article of La Russa & Qi (25); see in particular Table 1. The most commonly used S. pyogenes Cas9 protein is encoded by a 4.2 kb gene, which is just within the packaging limit of AAV vectors. Recently, an even smaller Cas9 orthologue was isolated from S. aureus and shown to have similar editing capabilities to the S. pyogenes Cas9, while its gene is 25% shorter. This facilitates packaging with a single guide RNA cassette into a single AAV vector. See also Dominguez et al. (26). Both review articles are incorporated herein in their entirety by reference. Further preferred embodiments of the expression construct are as described above.

In still a further embodiment, TBX5 expression can be enhanced by inactivating endogenous inhibitors of said TBX5 expression. For example, micro-RNA, such as micro-RNA-10a has recently been identified as a repressor of Tbx5 (Wang et al. (27); incorporated herein by reference). In said publication, expression of TBX5 and microRNA-10a was altered by cell transfection of siRNA or miRNA inhibitor, i.e. an antagomir. See, for example, FIGS. 2C and 2D therein. Antagomirs also known as anti-miRs or blockmirs are a class of chemically engineered oligonucleotides that prevent other molecules from binding to a desired site on an mRNA molecule. Antagomirs are used to silence endogenous microRNA (miR). Hence, in another preferred embodiment, the expression construct encodes and/or is capable of expressing an inhibitor of a micro-RNA, e.g. a microRNA-10a inhibitor, preferably wherein said microRNA-10a inhibitor is selected from an antagomir and a siRNA. Further preferred embodiments of the expression construct are as described above.

In rodents, gene delivery, in particular by AAV1 and AAV6 appear more suitable for dardiac gene transfer through intramyocardial, intrapericardial, or intravascular (intracoronary) routes, while AAV8 and AAV9 can achieve more efficient cardiac transduction only via the transvascular route. In the procine model, genes were delivered by AAV1-mediated gene transfer by intramuscular injection, or by application to the left anterior descending artery.

However, in a canine model, direct delivery of adenoviral vectors into coronary arteries resulted in relatively low myocardial gene expression, and high viral titers were required carrying the worrisome risk of undesired effects in tissues other than the myocardium. Such systemic contamination can be limited by use of an alternative catheter-based gene delivery device using pericardial application of adenoviral vectors. In these cases gene expression was limited to the parietal and visceral pericardium. Therefore, the pericardial approach does not yet seem to be sufficient to provide myocardial gene expression with homogenous transmural expression. These obstacles can be overcome by percutaneous transluminal retrograde gene delivery (PTRGD) through the coronary veins. See Boekstegers & Kupatt(28), which is incorporated herein by reference. Table 1 therein recites further articles in which genes have been delivered by retroinfusion. In another porcine model, MV particles (AAV9) were transferred successfully in a retrograde fashion via the cardiac anterior interventricular vein during blocking the venous outflow and the left anterior descending artery arterial inflow (cf. Katz et al.(23); supra). Hence, in a preferred embodiment, the expression construct is administered by coronary venous retroinfusion, preferably by percutaneous transluminal retrograde gene delivery (PTRGD) or retroinfusion via Sinus venosus.

Independent of the delivery route (intracoronary, retrograde into the cardiac venous system, or intramuscular) a high vector spillover into the system is often observed after first passage throught the cardiac vasculature. In order to avoid such effects, ultrasound-targeted microbubble destruction (UTMD) can be used to deliver the expression construct for use of the present disclosure to the myocardium. Thus, in a preferred embodiment, the expression construct is administered by ultrasound-targeted microbubble destruction (UTMD). Said method uses local application of the oscillatory effects of ultrasound on microbubbles at their resonance frequency to allow regional specific destruction of the bubbles. A method describing AAV6-mediated gene transfer by retroinfusion of the anterior interventricular vein of AAV6 mounted to microbubbles is exemplified in Schlegel et al. ((29), incorporated herein by reference), in particular on page 72, the paragraph bridging the columns. Further guidance is provided in Dimcevski et al. (30; incorporated herein in its entirety by reference. In another embodiment contemplated herein, the expression construct is administered by protein transduction domains (PTDs). PTDs are powerful nongenetic tools that allow intracellular delivery of conjugated cargoes to modify cell behaviour. Dixon et al. shows that a fusion protein that couples a membrane docketing peptide to heparin sulphate glycosaminoglycans (GAGs) with a PTD could deliver nucleic acids such as vectors, DNAs, cDNAs, RNAs, modified RNAs and siRNAs, but also transcription factors (such as TBX5) at high efficiencies in cell types hard to transduce. See Dixon et al. (31) for details, which is incorporated herein in its entirety by reference.

In light of the teaching of Dixon et al. (31), it is further plausible to directly deliver TBX5 protein to the ventricular cardiomyocytes. Therefore, it is further contemplated TBX5 protein for use in the prevention or monotherapeutic treatment of a ventricular heart disease and associated complications selected from cardiac arrhythmia and sudden cardiac death, wherein the TBX5 is delivered using protein transduction domains, in particular using a fusion as described by Dixon et al. (31). Likewise disclosed is the use of TBX5 protein formulated with or fused to a protein transduction domain, in particular using a fusion as described by Dixon et al. (31), in the preparation of a medicament for the prevention or monotherapeutic treatment of a ventricular heart disease and associated complications selected from cardiac arrhythmia and sudden cardiac death. Further disclosed is a method for the prevention or monotherapeutic treatment of a ventricular heart disease and associated complications selected from cardiac arrhythmia and sudden cardiac death, wherein the method comprises the step of administering a subject to be treated TBX5 protein formulated with or fused to a protein transduction domain, in particular using a fusion as described by Dixon et al. (31).

The invention is further described in the following Figures and Examples, which are not to be construed to limit the invention. The scope of the invention is defined by the claims only.

### DESCRIPTION OF THE FIGURES

FIG. 1: TBX5 expression in human and mouse left ventricles. Immunoblot analysis of human left ventricles with dilated (DCM) and ischemic cardiomyopathy (ICM) shows reduced expression of TBX5 compared to non-failing (NF) samples when normalized to CASQ2 or GAPDH. Samples loaded on the same blot, but non-contiguously are indicated by a black line.

FIG. 2. Characteristics of vTbx5KO model. (A) Primers for recombination qRT-PCR are designed to bind inside of Exon 3 and thereby allow specific detection of the missing exon. (B) qRT-PCR reveals that TBX5 recombination occurs in the ventricles and not in the atria (n=5, Flox samples; n=6, KO samples) Statistics; B was tested with student's t-test, *-p<0.05

FIG. 3. Characterization of vTbx5KO mice cardiac function under basal and stress conditions. (A) Mating scheme for vTbx5KO mouse generation from Myh6-MerCreMer⁹ mice and TBX5^{LDN/LDN 8} mice. (B) Survival curve of vTbx5KO mice shows significantly reduced lifespan as compared to control mice. (C) vTbx5KO are presented with contractile dysfunction with preserved ejection fraction EF at 8 weeks post-rec. as indicated by diastolic volume (vol d) and cardiac output (CO) decrease. 16 weeks post-rec. EF presents a mild but significant reduction. No hypertrophy observed as depicted by the heart weight/body weight (HW/BW) ratio. (D) Angiotensin II treated vTbx5KO mice show exacerbated cardiac function (EF), hypertrophic remodeling (HW/BW, LVPWth) and decompensation (LVd) as compared to angiotensin treated Flox mice. (E) CM Cross-sectional area (CSA) is increased in Ang treated vTbx5KO mice compared to Ang-Flox mice. (F) Collagen staining with Sirius Red shows that Ang-induced fibrosis is exacerbated in vTbx5KO vs Flox mice. Statistics; p-value-summary: *-<0.05. B, logrank test (Mantel-Cox); C, paired t-tests; (D-F)—One-way ANOVA followed by Sidak's multiple comparison test.

FIG. 4. Basal functional characterization of vTbx5KO mice vs Cre control. (A) Echocardiography data from vTbx5KO mice supporting contractile dysfunction show shorter left ventricle diameter (Ld), resulting in reduced stroke volume (SV). (B) vTbx5KO body weight increased over time, while heart weight slightly decreased. Heart rate (HR) upon TBX5 loss was not significantly altered. (C) Cre controls show no decrease in cardiac function as shown by EF, CO. No hypertrophy or hypotrophy was observed in Cre controls as depicted by HW/BW ratio. No HR changes occurred. (D) No significant fibrosis in the KO animals compared to controls was observed.

FIG. 5. vTbx5KO mice presented with conduction defects and arrhythmia. (A) Representative ECG traces of Flox and vTbx5KO mice recorded by telemetric ECG 2 weeks upon recombination. (B) Statistical analysis of telemetric ECG measurements reveals prolonged PR and QRS-intervals from 1-8 weeks post-rec. Line indicates Cre control mean value±SEM 4 weeks post-rec. (C) vTbx5KO mice present with atrioventricular blocks, ventricular tachycardias and asystoles. (D) Electrophysiological studies of isolated paced Flox, Cre and vTbx5KO hearts show prolonged activation times from RA to RV, endocardial RV to epicardial RV, RV to septum and RV to LV. Statistics; p-value-summary: *-<0.05. B, one-way ANOVA with Sidak's multiple comparison test against pre-recombination data of the same group; n=4-13/group. C, One-way ANOVA followed by Tukey's multiple comparison test

FIG. 6. vTbx5KO hearts are more arrhythmogenic as compared to controls. Ex Vivo burst pacing induced arrhythmia occurrence is higher (60%) in vTbx5KO mice vs Flox (23%) and Cre (38%) mice. Statistics for A+B: paired student's t-test against the pre time point. p-value-summary: *-<0.05

FIG. 7. Heart-specific re-expression of TBX5 leads to robust expression of TBX5 in the ventricle. (A) TBX5 is specifically expressed in the heart, not in liver and spleen. Band A—overexpressed TBX5-flag, B—unspecific band and C—endogenous TBX5. TBX5 was detected with the anti-TBX5 (HPA008786) (B) Immunofluorescence staining for TBX5 with the anti-TBX5 (HPA008786), Scale bar: 20 μm (C) PR-interval is slightly shorter in KO-RE group.

FIG. 8. In vivo TBX5 re-expression rescues arrhythmic phenotype of vTbx5KO mice while restoring TBX5 mediated transcription. (A) Transcript level of Tbx5 in KO-CT mice (left) and KO-RE mice (right) analyzed by qPCR. Shown is the relative mRNA expression normalized to Gapdh. (B) Heart-rate-variability (HRV) represented by Poincaré plots; low variability in KO-RE indicates lower incidence of arrhythmia compared to KO-CT. 1000 consecutive beats were included per mouse/plot. (C) The Poincaré plots were statistically analyzed, the standard deviation of the HRV (SD1) was clearly increased in KO-CT mice (suggestive of arrhythmias) and remained comparable to Cre control mice after AAV9-transduction (KO-RE). Dashed lines indicated Cre mean values±SEM. (D) Statistical analysis of SD1 from HRV analysis, shows significantly lower HRV in KO-RE vs KO-CT mice. (n=3-5 per group) (E) QRS prolongation in the KO-RE mice is partially reversed after TBX5 re-expression. Statistics; A,B,F,G, unpaired, two-tailed students-t-test; * p<0.05

### EXAMPLES

Materials and Methods

Study Design

The objective of this study was to determine the impact TBX5 loss in electrical signal propagation in the adult ventricles and to test the therapeutic potential of its re-expression. Human heart failure samples vs non-failing controls were used to determine TBX5 expression in human diseased hearts. The investigation conforms to the principles outlined in the Declaration of Helsinki. The study was approved by the institutional ethics committee. Inducible, cardiac specific TBX5 knock-out (vTbx5KO) and genotype control Myh6-MerCreMer and Tbx5LDN/LDN models were used to investigate the impact of TBX5 loss in the ventricle. Sample size was chosen based on GPower 3.1 calculation after pilot studies. Echocardiographic and telemetric electrocardiographic analyses were performed by the SFB 1002 service unit (S01 Disease Models). The observer was unaware of the genotypes and treatments. All animal experiments were approved by the Niedersachsen (AZ-G15/2029) animal review board. Echocardiographic and intervention details are described in Supplementary Materials and Methods.

Animal Experiments

Tbx5^{LDN/LDN} mice (15) were crossed with Myh6-MerCreMer (9) deleter mice in a C57BL/6N background. Activation of Cre-recombinase was induced by i.p. injections of tamoxifen (TMX) for three subsequent days (30 mg/kg/day [Sigma Aldrich, Hamburg/Germany], dissolved in 10% Ethanol [Carl Roth, Karlsruhe/Germany] and 90% Miglyol [Caelo, Hilden/Germany]). The inventors denoted the recombined mice as vTbx5KO, Tbx5^{LDN/LDN} mice as Flox, and Myh6-MerCreMer deleter mice as Cre throughout the study. Irrespective of the genotype, all animals were injected with TMX to control for TMX-induced effects. Recombination was confirmed by qPCR using a primer pair flanking exon3 and exon 4 of TBX5 transcript (FIG. 2A).

For hypertrophy induction, 2 weeks upon TMX injections, osmotic minipumps (Alzet) were implanted in vTbx5KO or Flox mice for the delivery of angiotensin II (Ang; 1.44 mg/kg/day for 2 weeks). For in vivo re-expression AAV vectors were injected into the tail vein 2 weeks upon TMX injections. All animal experiments were approved by the local competent authority (Niedersachsisches Landesamt fur Verbraucherschutz and Lebensmittelsicherheit—LAVES; AZ-G15/2029).

Echocardiography Analysis

Echocardiography was performed in anesthetized mice, under 2% isoflurane inhalation, as described previously (16). Ventricular dimensions were measured with a Visual Sonics Vevo 2100 Imaging System equipped with a 45 MHz MS-550D MicroScan transducer. The observer was unaware of genotype and treatment. All procedures were performed by the SFB 1002 service unit (S01 Disease Models) according to standard operating procedures.

Electrophysiological Study of Isolated Hearts

Hearts were excised under deep terminal anesthesia, and the aorta was cannulated and retrogradely perfused using 37° C. Krebs-Henseleit buffer (in mmol/l; NaCl 118, NaHCO₃ 24.88, KH₂PO₄ 41.18, glucose 5.55, Na-pyruvate 2, MgSO₄ 0.83, CaCl₂ 1.8, KCl 4.7) is equilibrated with a 95% oxygen/5% carbon dioxide gas mixture. The hearts were mounted on a vertical Langendorff apparatus (Hugo Sachs Electronic-Harvard Apparatus GmbH) and constantly perfused. An octapolar mouse electrophysiologic catheter (CIBER MOUSE; NuMED) was placed in the right atria and ventricles for atrial and ventricular pacing. Three murine monophasic action potentials (MAPs) were continuously and simultaneously recorded from the right ventricular free wall, septal area and left ventricular free wall epicardium (17). Atrial S1 pacing was performed to measure activation times from right atrium to right ventricle; endocardial right ventricular pacing was performed to measure ventricular activation times and both for steady-state action potential durations. To test ventricular arrhythmia inducibility, programmed ventricular stimulation was performed using a single encroaching premature stimulus (S2). All signals were digitized and stored on digital media for offline analysis. Experiments and analysis were performed in a blinded fashion. Details of the method have been described previously (18).

RNA Isolation, Reverse Transcription, and Quantitative PCR Analysis

RNA was isolated using the NucleoSpin® RNA kit (Macherey-Nagel, Dueren/Germany) according to the manufacturer's instructions. Reverse transcription and quantitative PCR (qRT-PCR) were performed as described previously (19). All primer sequences used in this study are listed in the following Table 1.

TABLE 1
Murine Primer Sequences

| Name | Use | Sequence 5′-3′ | SEQ ID NO: |
|---|---|---|---|
| TBX5rec forward | qRT-PCR | AGGCAGGGAGGAGAATGTTT | 1 |
| TBX4rec reverse | qRT-PCR | GGCTCTGCTTTGCCAGTTAC | 2 |
| Gapdh forward | qRT-PCR | ATGTTCCAGTATGACTCCACTCACG | 3 |
| Gapdh reverse | qRT-PCR | GAAGACACCAGTAGACTCCACGACA | 4 |
| Tbx5-CDS forward | cloning | GCTATAGAATTCTGGCCGATACAGATGAGGG | 5 |
| Tbx5-CDS reverse | cloning | TATAGTCGACGCTATTCTCACTCCACTCTG | 6 |

AAV Vector Production:

MV serotype 9 vector production and purification was done according to Jungmann et al.(20). In short, the helper plasmid pDP9rs (a derivate of pDP2rs) and an MV vector genome plasmid (either pdsTnT-rluc or pds-TnT-mTBX5) were co-transfected into 293T cells resulting in AAV9-luc or AAV9-TBX5. pds-TnT-rluc contains a Renilla luciferase reporter gene and pdsTnT-mTBX5 contains the murine cDNA of TBX5 both under control of the −502/+42 bp human troponin-T promoter (Tnnt2) (21). MV vectors were purified using Iodixanol step gradient centrifugation and titrated as reported before (20.

Statistical Analysis

Differences between experimental groups were analyzed using one-way ANOVA followed by appropriate post hoc test as indicated in the Figure legends when more than 2 groups were compared, or student's t-test if the assay contained only two groups. Data are presented as individual data points with mean (indicated by a horizontal line), bar graphs with standard error of the mean (SEM), or as box-and-whiskers-plots with the box extending from the 25^{th} to 75^{th} percentile, whiskers indicating min to max, + indicates the mean value and the line indicates the median. In the manuscript values are presented as mean±SEM. p<0.05 values were considered significant.

Study Approval

All experimental procedures to which mice were subjected were approved by the animal review board LAVES (Niedersachsiches Landesamt fur Verbraucherschutz and Lebensmittelsicherheit; 15/2029) or UK home office (30/2967).

All DCM, ICM and non-failing patient samples were collected according to an approval by the responsible Ethics Committee at the University Medical Center Gottingen (31/9/00).

Telemetric ECG Analysis

Mice were implanted with telemetric ECG transmitters ETA-F10 (Data Science International) subcutaneously as described before (22). Mice were allowed to recover and stabilize for 2 weeks prior to any intervention. 24 hours ECGs were recorded before KO induction with TMX and 1, 2, 4 and 8 weeks after recombination. ECG recordings were analyzed with Ponemah Physiology Platform 6.3 (Data Science International) using template based analysis.

### Example 1—TBX5 Protein is Reduced in Failing Human Myocardium

Whereas the role of TBX5 in congenital heart disease has been well documented in humans and rodents (6), a contribution of TBX5 to the homeostasis of ventricular myocardium in the adult heart remains elusive. Hence, the inventors first studied TBX5 expression in samples obtained from the left ventricles of non-failing (NF) hearts as well as heart explants from patients with ischemic (ICM) and dilated cardiomyopathy (DCM); all patients presented with arrhythmias such as ventricular tachycardia and atrial fibrillation (Table 2).

TABLE 2
DCM and ICM Patient characteristics.

| | | |
|---|---|---|
| DCM | ICM | NF |

| | | | |
|---|---|---|---|
| n | 4 | 4 | 3 |

| | | | | | |
|---|---|---|---|---|---|
| Age, mean (SD) | 54 | (10) | 52.5 | (4) | n/a |

| | | | |
|---|---|---|---|
| Age, range | 40-65 | 47-57 | n/a |

| | | | | | |
|---|---|---|---|---|---|
| EF, mean (SD) | 22.3% | (10) | 26.25% | (8) | n/a |

| | | | |
|---|---|---|---|
| EF, range | 15-40% | 20-40% | n/a |
| Sex | | | |

| Male, percentage (n) | 75% | (3) | 100% | (4) | n/a |
|---|---|---|---|---|---|
| Female, percentage (n) | 25% | (1) | | | n/a |
| History of arrhythmia | 100% | (4) | 100% | (4) | n/a |

n/a not available

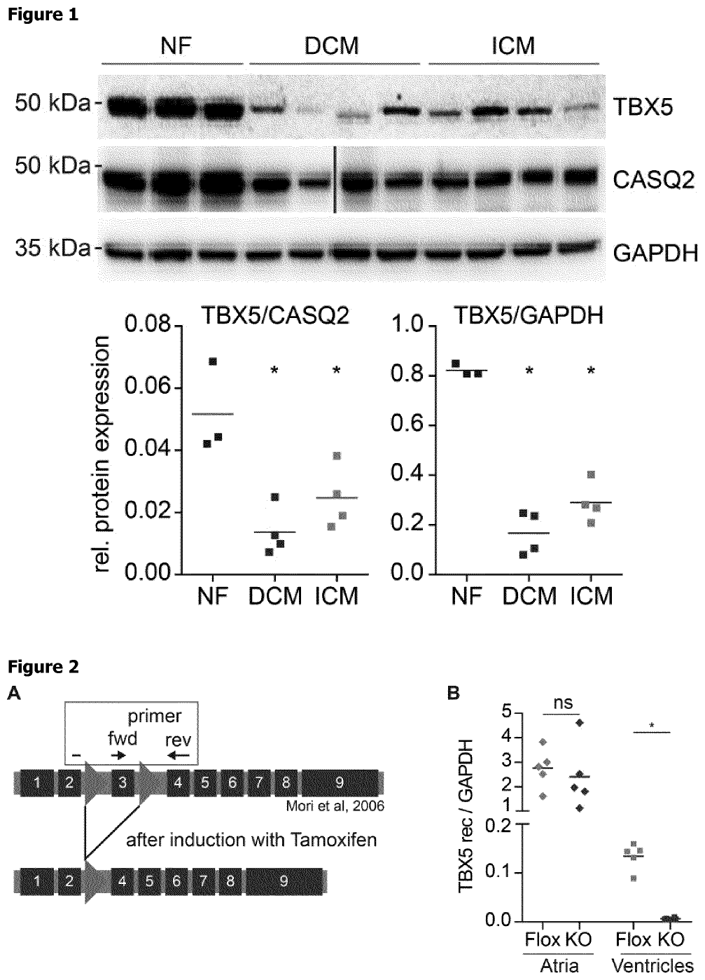

The inventors found TBX5 protein to be of significantly lower abundance in ICM and DCM versus NF heart muscle, suggesting a role for TBX5 beyond the context of congenital heart disease (FIG. 1).

Ventricular TBX5 Expression is Essential for Maintaining Normal Adult Cardiac Homeostasis

Previous reports showed that in the adult mouse heart TBX5 is strongly expressed in the atria and the VCS (6). The inventors confirmed this finding by qPCR and noted that TBX5 mRNA in ventricular myocardium was ˜5% of the atrial expression (FIG. 2B).

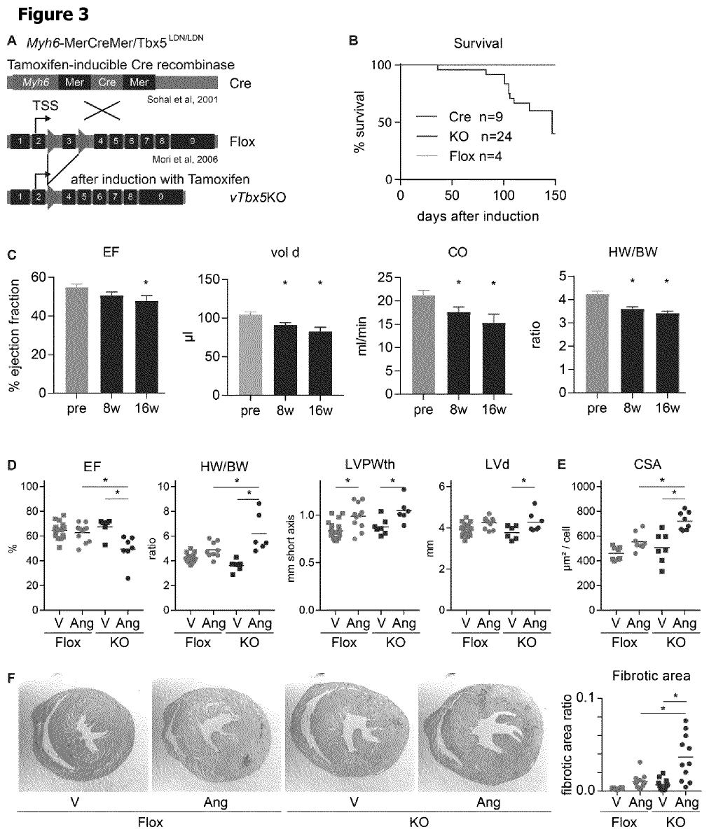

To investigate the role of TBX5 in the adult heart, the inventors generated a conditional TMX-inducible CM-specific knock-out mouse model (Myh6-MerCreMer/TBX5^{LDN/LDN}) by mating two established mouse models (FIG. 3A) (5, 9). TMX-induced recombination occurred in the ventricles, but not in the atria (FIG. 2B), serendipitously providing a ventricular-specific Tbx5 KO model (denoted as vTbx5KO). Inefficient recombination in the atria of mice mated with Myh6-MerCreMer has been reported earlier (10, 11).

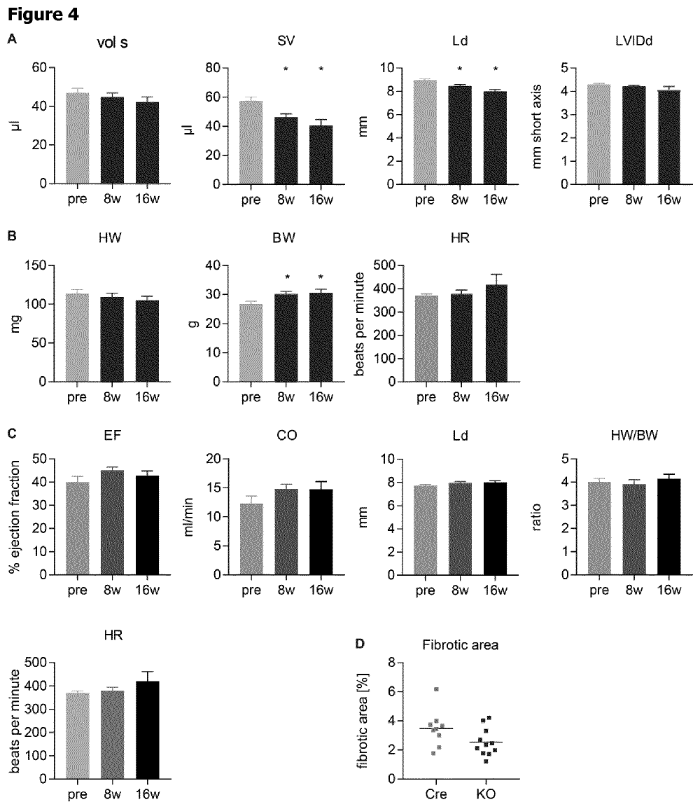

TBX5 loss in ventricular cardiomyocytes significantly impacted animal survival 3-4 months upon recombination (FIG. 3B). Echocardiography analysis revealed progressive dysfunction (FIG. 3C, 4A) 8 weeks upon recombination which resulted in moderate cardiac function deterioration by 16 weeks (Δ 7.6±2.4% in ejection fraction (EF), p<0.05 in vTbx5KO FIG. 3C, 4A). A similar phenotype is observed in HOS patients as well as in Tbx5 haploinsufficient mice (12). As a result, cardiac output was significantly lower in vTbx5KO while heart rate was not reduced (FIG. 3C, 4A, 4B). In contrast to vTbx5KO, Cre control mice maintained a normal cardiac function and structure (FIG. 4C). Interestingly, vTbx5KO heart mass did not increase with aging (FIG. 4B) resulting in a decreased heart weight to body weight ratio (HW/BW 22% less, ±3.1%, FIG. 3C). Sirius red stains in Cre and vTbx5KO hearts did not show evidence for fibrosis (FIG. 4D).

vTbx5KO Leads to Accelerated Cardiac Decompensation Upon Remodeling

To study the role of TBX5 upon cardiac remodeling, the inventors induced mild hypertrophy by chronic stimulation with Ang in vTbx5KO and Flox mice. 4 weeks upon recombination, Flox and vTbx5KO mice did not show differences in cardiac function (FIG. 3D), hypertrophic growth (FIG. 3D, 3E), and fibrosis (FIG. 3F). However, under Ang treatment vTbx5KO mice developed heart failure with reduced EF (A 14±4%, p<0.05, FIG. 3D) accompanied with an exacerbated hypertrophic and fibrotic response (FIG. 3D-F). Although Ang treatment induced hypertrophy in both Flox and vTbx5KO mice (LVPWth increase of 0.15±0.4 mm and 0.17±0.06 mm respectively, p<0.05, FIG. 3D), only the latter exhibited left ventricular dilation (LVd increase of 0.5±0.18 mm, p<0.05, FIG. 3D), indicating an earlier onset of decompensation in the absence of TBX5.

TBX5 Expression is Essential for Electrical Signal Propagation in the Adult Ventricle

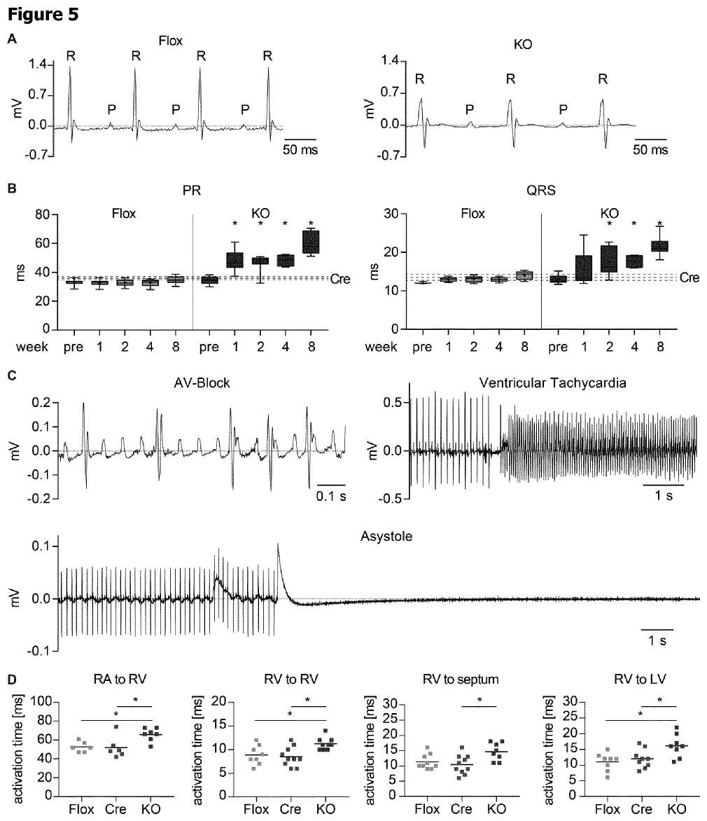

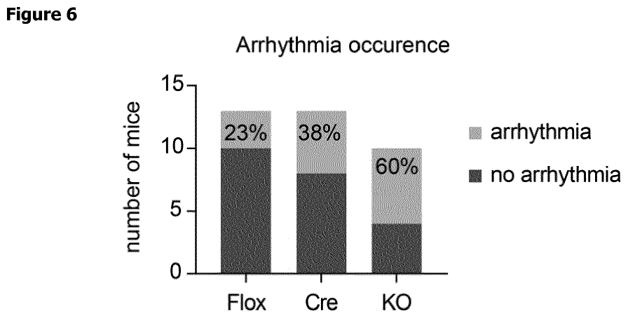

To evaluate the impact of TBX5 loss on cardiac conduction, the inventors monitored cardiac rhythm by telemetric ECG analysis a day prior (0 week) and 1, 2, 4 and 8 weeks post-recombination. This revealed a significant PR and QRS prolongation one week (FIG. 5A,B) and two weeks upon recombination in the vTbx5KO mice, respectively. Flox and Cre controls had no significant changes in any of the measured ECG parameters. 8 weeks upon recombination PR and QRS interval were prolonged by 26±2 and 7.5±1.2 ms, respectively. Moreover, all vTbx5KO mice presented 2^{nd} degree atrioventricular blocks, which is in line with previously published data (5) and suggests that the AV node is also affected in the vTbx5KO model. The inventors often also recorded 3^{rd} degree AV blocks with ventricular escape rhythm, ventricular tachyarrhythmia and occasionally asystole (FIG. 5C). In line with the inventors' findings, HOS patients with TBX5 loss-of-function presented with AV blocks and ventricular tachycardia (13). The increased propensity for ventricular arrhythmia was further substantiated by right ventricular endocardial septal S1S2 pacing in isolated hearts from Flox, Cre, and vTbx5KO mice, showing that arrhythmias in 60% of the vTbx5KO vs. 23% and 38% in Flox and Cre hearts, respectively (FIG. 6).

S1 pacing in isolated hearts with an octapolar catheter showed a prolonged electrical propagation from the RA to RV in vTbx5KO (13.5±4.6 ms vs. Cre/Flox; FIG. 5D), in line with the PR prolongation observed by telemetry and in agreement with the findings reported earlier in a VCS-specific TBX5-KO model (6). The prolongation of electrical activation time from the RV septum to the epicardial free wall (2.5±0.8 ms vs. Cre/Flox), from the RV to the ventricular septum (3.5±1.4 ms vs. Cre/Flox) and from the RV endocardium to the LV of vTbx5KO hearts (4.5±1.6 ms vs. Cre/Flox) extended these findings and supported the inventors' hypothesis that TBX5 is a critical and so far underappreciated control element for the regulation of electrical activation in the working myocardium.

TBX5 Re-Expression Restores Related Transcriptional Profiles and Rescues the Arrhythmia Phenotype in vTbx5KO Mice

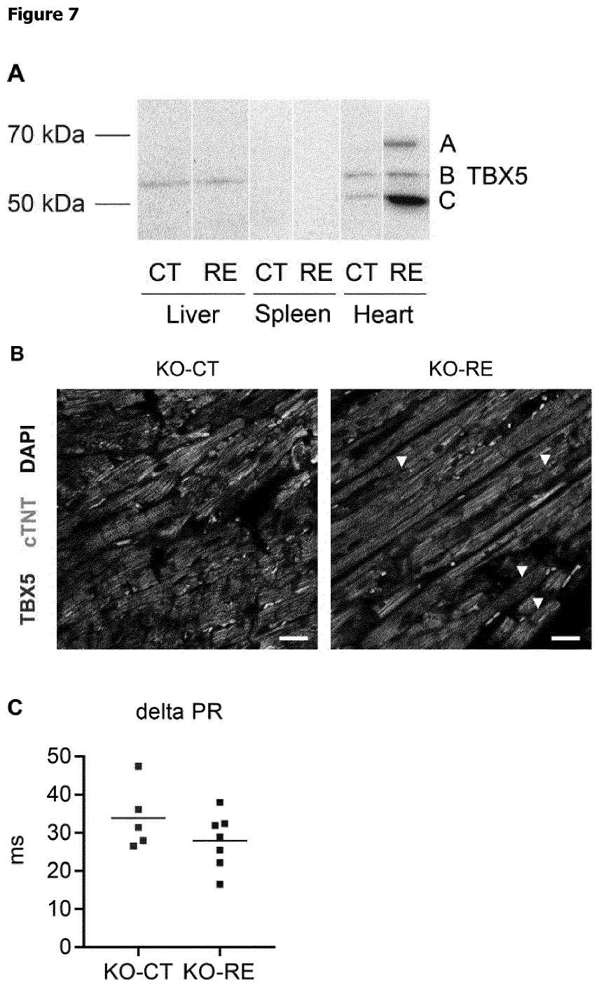

Our data showed that ventricular suppression of TBX5 is detrimental to the adult heart due to transcriptional dysregulation of important cardiac genes implicated in arrhythmia and SCD. Thus, the inventors asked whether TBX5 re-expression could restore TBX5-mediated transcription and thus rescue the arrhythmia phenotype. To address this question, the inventors injected vTbx5KO mice (2 weeks upon recombination) with AAV9 vectors (2×10¹² per mouse) containing the coding sequence of TBX5 (KO-RE, n=7) under the Tnnt2 promoter or a control vector (KO-CT, n=6). 6 weeks upon AAV9 injections, mice were sacrificed and the transcript levels of TBX5 and its target genes were quantified. AAV9-TBX5 injection resulted in cardiac-specific TBX5 re-expression (FIG. 7A, 7B).

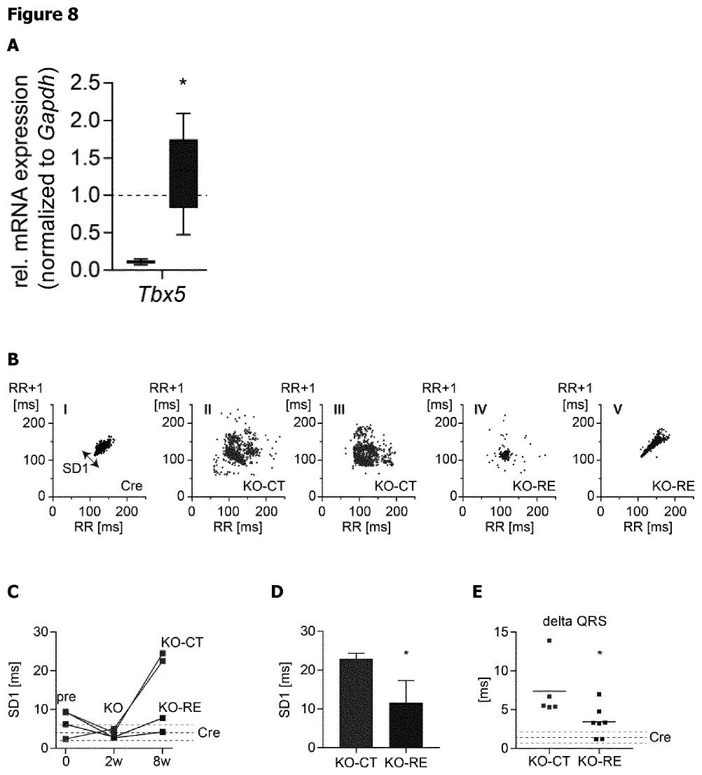

To evaluate the therapeutic potential of TBX5 to reverse arrhythmias observed in KO mice the inventors analysed RR tachography by Poincaré plots comprising RR data from prior to recombination (pre) and 2 weeks upon recombination (KO), but prior to AAV9 delivery and 6 weeks upon delivery of AAV9 encoding for a luciferase control gene (KO-CT) or TBX5 (KO-RE). 2 weeks upon recombination, all mice had a similar RR variability depicted by similarly low standard deviation 1 (SD1; 4.9±0.8 ms), suggesting low or no arrhythmias. However, during the course of 6 weeks, KO-CT mice severely deteriorated (SD1 23±1.4 ms) whereas KO-RE mice with TBX5 re-expression presented with a more regular RR (SD1: 11.6±5.7 ms) similar to the Cre control mice (SD1: 4.3±2 ms; FIG. 8B-D). Since vTbx5KO mice presented with a prominent PR and QRS interval prolongation (ΔPR and ΔQRS), the inventors examined whether TBX5 re-expression can restore normal conduction. ΔQRS was significantly less in KO-RE mice compared to KO-CT (3.4±0.8 ms vs. 7.4±1.7 ms; FIG. 8E) while ΔPR did not reach statistical significance (FIG. 7C) possibly due to the variation of Tbx5 re-expression levels between the mice (FIG. 8A).

These data provide proof of principle that normalization of TBX5 level is able to reduce arrhythmias by restoring TBX5 mediated transcription.

Discussion

TBX5 is an essential transcription factor for normal cardiac development. Mutations in the TBX5 locus are linked to abnormal cardiac conduction (Holt Oram Syndrome). The role of TBX5 in the atria and the VCS has been investigated in detail (6, 7). On the contrary, due to the relatively low TBX5 levels in the ventricular myocardium it was believed that its role in this cardiac compartment may be less important.

Our study demonstrates that ventricular TBX5 is suppressed in human HF, suggesting that its loss may affect cardiac pathologies beyond congenital heart disease. To investigate the impact of ventricular TBX5 loss in the adult heart, the inventors generated a Myh6-MerCreMer TBX5 model with no apparent TBX5 recombination in the atria (vTbx5KO). Inefficient recombination in the atria of genetically modified mice mated with Myh6-MerCreMer has been reported earlier (10, 11). At baseline, vTbx5KO mice presented with progressive dysfunction similar to what is observed in TBX5 haploinsufficient mice (6, 12).

Since TBX5 is known to play an important role in cardiac conduction, the inventors investigated if the impact of its loss in the whole ventricle would lead to a more severe phenotype than the one observed in VCS specific TBX5 deletion (6). Indeed, the delay in electrical signal propagation in the ventricles (QRS) was 70% more pronounced as compared to the VCS TBX5KO model (6). This may be a result of the significantly longer activation times observed in the ventricular myocardium of vTbx5KO mice. Moreover, the earlier onset of SCD and the higher number of SCD affected vTbx5KO mice compared to the VCS TBX5KO model (6), strongly support the importance of TBX5 in ventricular cardiomyocytes.

To characterize the role of TBX5 in the stressed heart, the inventors chronically challenged vTbx5KO and control mice with Ang. vTbx5KO mice presented with exacerbated remodelling and functional deterioration as compared to the respective controls.

Previous studies have depicted the importance of TBX5 in the developing heart but also in the adult atria and VCS(2, 3, 5-7, 12, 15). In this study, the inventors show for the first time that ventricular TBX5 levels are particularly low in heart failure patients suffering from arrhythmias, rendering TBX5 as an interesting therapeutic target against arrhythmia development. Thus, the inventors tested the potential of TBX5 re-expression to reverse arrhythmia phenotype in vTbx5KO mice that already developed a ventricular conduction delay. Despite the TBX5 re-expression level variability between mice, the inventors' data provide proof of concept that TBX5 enhancement can reduce related electrical signal propagation delay and the heart rate variability observed in vTbx5KO mice.

In conclusion, low TBX5 expression in DCM and ICM human ventricles suggested a dysregulation of TBX5 in HF patients. Ventricular KO of TBX5 in the adult murine heart resulted in contractile dysfunction, electrical signal propagation delay and arrhythmias with a consequent incidence of SCD. Upon mild hypertrophic stimulus, vTbx5KO mice presented exacerbated cardiac dysfunction and remodeling as compared to the respective controls. In line with these phenotypes, the inventors identified novel downstream TBX5 targets in the mouse ventricles involved in cardiac conduction, cytoskeleton organization and cardioprotection. Finally, in vivo TBX5 re-expression restored TBX5-mediated transcription is and rescued the arrhythmic phenotype. Collectively, the inventors' data provide proof-of-concept for the therapeutic potential of the restoration of TBX5-related transcription to reduce arrhythmias in the failing heart.

### LIST OF REFERENCES

1. A. S. Adabag, R. V. Luepker, V. L. Roger, B. J. Gersh, Sudden cardiac death: epidemiology and risk factors. Nat Rev Cardiol 7, 216-225 (2010).
2. M. E. Horb, G. H. Thomsen, Tbx5 is essential for heart development. Development 126, 1739-1751 (1999).
3. C. T. Basson et al., Mutations in human TBX5 [corrected] cause limb and cardiac malformation in Holt-Oram syndrome. Nature genetics 15, 30-35 (1997).
4. H. Holm et al., Several common variants modulate heart rate, PR interval and QRS duration. Nature genetics 42, 117-122 (2010).
5. B. G. Bruneau et al., A murine model of Holt-Oram syndrome defines roles of the T-box transcription factor Tbx5 in cardiogenesis and disease. Cell 106, 709-721 (2001).
6. D. E. Arnolds et al., TBX5 drives Scn5a expression to regulate cardiac conduction system function. The Journal of clinical investigation 122, 2509-2518 (2012).
7. R. D. Nadadur et al., Pitx2 modulates a Tbx5-dependent gene regulatory network to maintain atrial rhythm. Science translational medicine 8, 354ra115 (2016).
8. A. D. Mori, B. G. Bruneau, TBX5 mutations and congenital heart disease: Holt-Oram syndrome revealed. Curr Opin Cardiol 19, 211-215 (2004).
9. D. S. Sohal et al., Temporally regulated and tissue-specific gene manipulations in the adult and embryonic heart using a tamoxifen-inducible Cre protein. Circulation research 89, 20-25 (2001).
10. T. Shen et al., Tbx20 regulates a genetic program essential to adult mouse cardiomyocyte function. J Clin Invest 121, 4640-4654 (2011).
11. P. C. Hsieh et al., Evidence from a genetic fate-mapping study that stem cells refresh adult mammalian cardiomyocytes after injury. Nat Med 13, 970-974 (2007).
12. Y. Zhu et al., Tbx5-dependent pathway regulating diastolic function in congenital heart disease. Proceedings of the National Academy of Sciences of the United States of America 105, 5519-5524 (2008).
13. C. J. Boogerd et al., Functional analysis of novel TBX5 T-box mutations associated with Holt-Oram syndrome. Cardiovascular research 88, 130-139 (2010).
15. A. D. Mori et al., Tbx5-dependent rheostatic control of cardiac gene expression and morphogenesis. Dev Biol 297, 566-586 (2006).
16. M. P. Zafiriou et al., Erythropoietin responsive cardiomyogenic cells contribute to heart repair post myocardial infarction. Stem Cells 32, 2480-2491 (2014).
17. L. Fabritz et al., Prolonged action potential durations, increased dispersion of repolarization, and polymorphic ventricular tachycardia in a mouse model of proarrhythmia. Basic Res Cardiol 98, 25-32 (2003).
18. K. Wittkopper et al., Constitutively active phosphatase inhibitor-1 improves cardiac contractility in young mice but is deleterious after catecholaminergic stress and with aging. The Journal of clinical investigation 120, 617-626 (2010).
19. M. P. Zafiriou et al., Hepoxilin A(3) protects beta-cells from apoptosis in contrast to its precursor, 12-hydroperoxyeicosatetraenoic acid. Biochim Biophys Acta 1811, 361-369 (2011).
20. A. Jungmann, B. Leuchs, J. Rommelaere, H. A. Katus, O. J. Muller, Protocol for Efficient Generation and Characterization of Adeno-Associated Viral Vectors. Hum Gene Ther Methods 28, 235-246 (2017).
21. S. Werfel et al., Rapid and highly efficient inducible cardiac gene knockout in adult mice using MV-mediated expression of Cre recombinase. Cardiovascular research 104, 15-23 (2014).
22. C. Vettel et al., Phosphodiesterase 2 Protects Against Catecholamine-Induced Arrhythmia and Preserves Contractile Function After Myocardial Infarction. Circulation research 120, 120-132 (2017).
23. Katz et al. Use of Adeno-Associated Virus Vector for Cardiac Gene Delivery in Large-Animal Surgical Models of Heart Failure; Human Gene Therapy Clinical Development, 28, 157-164 (2017).
24. Lee et al. Promoter-specific lentivectors for long-term, cardiac directed therapy of Fabry disease, Journal of Cardiology, 57, 115-122 (2011).
25. La Russa & Qi, The New State of the Art: Cas9 for Gene Activation and Repression, Molecular and Cellular Biology 35, 3800-3809 (2015).
26. Dominguez et al. Beyond editing: repurposing CRISPR-Cas9 for precision genome regulation and interrogation, Nature Reviews Molecular Cell Biology, Volume 17, 5-15 (2016).
27. Wang et al. microRNA-10a targets T-box 5 to inhibit the Development of Cardiac Hypertrophy, Int Heart J 58: 100-106 (2017).
28. Boekstegers & Kupatt Basic Res Cardiol 99, 373-381 (2004).
29. Schlegel et al. Locally Targeted Cardiac Gene Delivery by AAV Microbubble Destruction in a Large Animal Model, Human Gene Therapy Methods 27, 71-78 (2016).
30. Dimcevski et al. A human clinical trial using ultrasound and microbubbles to enhance gemcitabine treatment of inoperable pancreatic cancer, Journal of Controlled Release 243, 172-181 (2016).
31. Dixon et al. Highly efficient delivery of functional cargoes by the synergistic effect of GAG binding motifs and cell-penetrating peptides, PNAS 114, E4112-E4114 (2017).
32. Zhou et al. A novel TBX5 loss-of-function mutation associated with sporadic dilated cardiomyopathy. International Journal Of Molecular Medicine 36, 282-288 (2015).
33. Lai et al. Adenovirus and adeno-associated virus vectors, DNA Cell Biol. 21, 895-913 (2002).
34. WO 2013/063305 A2.
35. WO 2013/173714 A2.
36. WO 2014/071199 A1.

1. A method for preventing or monotherapeutically treating a ventricular heart disease and associated complications selected from cardiac arrhythmia and sudden cardiac death with an expression construct capable of enhancing expression of TBX5 in a subject to be treated.

2. The method of claim 1, wherein the subject to be treated suffers from heart insufficiency.

3. The method of claim 1 wherein the subject to be treated shows a reduced expression of TBX5 in ventricular cardiomyocytes.

4. The method of claim 1, wherein the subject is a mammal, preferably wherein the subject is a human.

5. The method of claim 1, wherein the expression construct is for normalizing the expression of TBX5 in ventricular cardiomyocytes.

6. The method of claim 1 wherein the expression construct is a recombined DNA, cDNA or RNA.

7. The method of claim 1, wherein the expression construct is a viral expression vector, preferably a lentiviral expression vector.

8. The method of claim 7, wherein the viral vector is an Adeno-associated viral (AAV) expression vector.

9. The method of claim 7, wherein the viral vector is an AAV serotype 6 vector, AAV serotype 9 vector, AAV serotype 1 vector, or AAV serotype 8 vector, preferably an AAV serotype 6 vector or AAV serotype 9 vector, more preferably an AAV serotype 6 vector.

10. The method of claim 1, wherein the expression construct comprises a cardiac-specific promoter, preferably wherein the cardiac-specific promoter is selected from cardiac troponin T promoter (cTnT), α-myosin heavy chain (α-MHC), myosin light chain (MLC2v).

11. The method of claim 1, wherein the expression construct comprises human cardiac troponin-T promoter.

12. The method of claim 1, wherein the expression construct encodes and/or is capable of expressing TBX5.

13. The method of claim 1, wherein the expression construct encodes and/or is capable of expressing a CRISPR-dCas9-activator system specific for Tbx5.

14. The method of claim 1, wherein the expression construct encodes and/or is capable of expressing a microRNA-10a inhibitor, preferably wherein said microRNA-10a inhibitor is selected from an antagomir and a siRNA.

15. The method of claim 1, wherein the expression construct is administered by coronary venous retroinfusion, preferably by percutaneous transluminal retrograde gene delivery (PTRGD) or retroinfusion via Sinus venosus.

16. The method of claim 1, wherein the expression construct is delivered by ultrasound-targeted microbubble destruction (UTMD).

17. The method of claim 1, wherein the expression construct is delivered by using protein transduction domains (PTDs).

18. A method for preventing or monotherapeutically treating of a ventricular heart disease and associated complications selected from cardiac arrhythmia and sudden cardiac death with a TBX5 protein, wherein the TBX5 protein is delivered using protein transduction domains.

19. The method of claim 18, wherein the protein transduction domain is a fusion of a GAG binding motif and a cell-penetrating peptide.


SEQUENCE LISTING (6 sequences)

SEQ ID NO: 1 - DNA, 20 bp, Artificial
  Forward Primer TBX5rec
aggcagggaggagaatgttt

SEQ ID NO: 2 - DNA, 20 bp, Artificial
  Reverse Primer TBX5rec
ggctctgctttgccagttac

SEQ ID NO: 3 - DNA, 25 bp, Artificial
  Forward Primer Gapdh
atgttccagtatgactccactcacg

SEQ ID NO: 4 - DNA, 25 bp, Artificial
  Reverse Primer Gapdh
gaagacaccagtagactccacgaca

SEQ ID NO: 5 - DNA, 31 bp, Artificial
  Forward Primer Tbx5-CDS
gctatagaattctggccgatacagatgaggg

SEQ ID NO: 6 - DNA, 30 bp, Artificial
  Reverse Primer Tbx5-CDS
tatagtcgacgctattctcactccactctg